(19) United States
(12) Patent Application Publication
(10) Pub. No.: US 2009/0234235 A1
(43) Pub. Date: Sep. 17, 2009

(54) SYSTEM AND METHOD OF TISSUE CLASSIFICATION

(75) Inventors: Dan HASHIMSHONY, Givat Ada (IL); Gil COHEN, Jerusalem (IL); Iddo GELTNER, Herzlia (IL); David BAR-MOSHE, Petach Tikva (IL)

Correspondence Address: OLIFF & BERRIDGE, PLC, P.O. BOX 320850, ALEXANDRIA, VA, 22320-4850, US

(73) Assignee: DUNE MEDICAL DEVICES LTD., Caesarea (IL)

(21) Appl. No.: 12/046,133

(22) Filed: Mar. 11, 2008

(51) Int. Cl. A61B 6/00; G06F 17/30

(52) U.S. Cl. 600/477; 600/476; 707/101; 707/E17.001

(57) ABSTRACT

Provided a method of automated classifying a tissue and a system thereof. The method comprises predefining a classification set comprising one or more classification parameters; acquiring data indicative of the tissue characteristics and sufficient for deriving values of said classification parameters; processing the acquired data and generating a value vector characterizing the tissue, said vector characterized by values of said classification parameters; matching the value vector to a plurality of predefined classification clusters in order to associate the tissue to be classified to an appropriate cluster, thus giving rise to a matching cluster; transforming the value vector into a score value by use of a transformation algorithm, wherein the transformation algorithm and/or parameters thereof are selected in accordance with the matching cluster; comparing the score value with a classification criterion corresponding to said matching cluster; and providing a classification decision in accordance with results of said comparing.

### FIELD OF THE INVENTION

The present invention relates to the field of medical devices, and, more particularly, to a system and method of tissue classification.

### BACKGROUND OF THE INVENTION

A large number of techniques and sensors are available today for tissue characterization, for example, to determine the presence of abnormal tissue, such as cancerous or pre-cancerous tissue. The problem of tissue characterization by applying different energy pulses and/or fields (e.g. electrical fringe field, polarizing magnetic field, RF impulses, etc.) and measuring and analyzing the respective electromagnetic or other properties of tissue (e.g. electric impedance, magnetic resonance, dielectric properties, optical characteristics, mechanical properties, etc.) has been recognized in prior art and various systems have been developed to provide a solution, for example:

U.S. Pat. No. 7,082,325 (Hashimshony et al.) entitled “Method and apparatus for examining a substance, particularly tissue, to characterize its type” discloses a method and apparatus for examining a substance volume to characterize its type, in particular, to characterize it as cancerous or non-cancerous. The method comprises applying a polarizing magnetic field through the examined substance; applying RF pulses locally to the examined substance volume such as to invoke electrical impedance (EI) responses signals corresponding to the electrical impedance of the substance, and magnetic resonance (MR) responses signals corresponding to the MR properties of the substance; detecting the EI and MR response signals; and utilizing the detected response signals for characterizing the examined substance volume type.

US Patent Application No. 2003/138,378 (Hashimshony.) entitled “Method and apparatus for examining tissue for predefined target cells, particularly cancerous cells, and a probe useful in such method and apparatus” discloses a method, apparatus and probe for examining tissue for the presence of target cells. Energy pulses are applied to the examined tissue. The changes in impedance and/or optical characteristics of the examined tissue produced by the applied energy pulses are detected and utilized for determining the presence of the target cells in the examined tissue.

US Patent Application No. 2007/179,397 (Hashimshony et al.) entitled “Method and system for examining tissue according to the dielectric properties thereof” discloses probes, systems, and methods for tissue characterization by its dielectric properties, wherein a physical feature of the probe is designed to define and delimit a tissue volume, at a tissue edge, where characterization takes place. Thus, the probe for tissue-edge characterization comprises: a first inner conductor, which comprises: proximal and distal ends, with respect to a tissue edge, along an x-axis; a first sharp edge, inherently associated with the proximal end; at least one feature, issuing from the first inner conductor, substantially at the proximal end, for forming at least one additional sharp edge, operative to enhance localized electrical fringe fields in the tissue, within a generally predefined tissue volume, at the tissue edge, the tissue volume being generally defined by physical parameters associated with the at least one feature; and a dielectric material, which encloses the conductor, in the y-z planes.

US Patent Application No. 2007/255,169 (Hashimshony et al.) entitled “Clean margin assessment tool” discloses an integrated tool, having a tissue-type sensor, for determining the tissue type at a near zone volume of a tissue surface, and a distance-measuring sensor, for determining the distance to an interface with another tissue type, for (i) confirming an existence of a clean margin of healthy tissue around a malignant tumor, which is being removed, and (ii) determining the depth of the clean margin. The integrated tool may further include a position tracking device and an incision instrument.

International Patent application WO07/83,310 (Hashimshony et al.) entitled “System and method for analysis and treatment of a body tissue” discloses a system and method for analysis and treatment of a tissue site. The system of the invention includes a probe unit containing one or more tissue sensing and monitoring probes configured to measure one or more parameters indicative of one or more states of the tissue site and one or more tissue treatment probes configured to deliver a treatment to the tissue site. A processor receives signals from the sensing and monitoring probes and determines whether the probe unit is located at the tissue site to be treated. The treatment and monitoring probes are activated in order to monitor the state of the tissue site while the treatment is being delivered to the tissue site. The processor receives signals from the sensing and monitoring probes during delivery of the treatment indicative of the state of the tissue site and determines, as the treatment is being carried out, any one or more of whether the treatment carried out so far is adequate, whether an additional treatment needs to be carried out, and whether the parameters of the treatment or the treatment targets need to be modified.

### SUMMARY OF THE INVENTION

The is a need in the art to provide a system and method for automated on-line tissue classification based on acquired data indicative of tissue parameters.

In accordance with certain aspects of the present invention, there is provided a method of automated classifying a tissue, the method comprising: predefining a classification set comprising one or more classification parameters; acquiring data indicative of the tissue characteristics and sufficient for deriving values of said classification parameters; processing the acquired data and generating a value vector characterizing the tissue, said vector characterized by values of said classification parameters; matching the value vector to a plurality of predefined classification clusters in order to associate the tissue to be classified to an appropriate cluster thus giving rise to a matching cluster; transforming the value vector into a score value by use of a transformation algorithm, wherein the transformation algorithm and/or parameters thereof are selected in accordance with the matching cluster; comparing the score value with a classification criterion corresponding to said matching cluster; and providing a classification decision in accordance with results of said comparing.

In accordance with other aspects of the present invention, there is provided a system for tissue characterization comprising at least one probe unit with one or more sensors, said probe unit operatively coupled to a classification unit configured to obtain data indicative of the tissue characteristics and measured by the probe unit with the help of said sensors, said classification unit comprising a processor operatively coupled to a data repository configured to accommodate a predefined classification set comprising one or more classification parameters; wherein the processor is configured to be capable of:
deriving from the obtained data values of said classification parameters, thus giving rise to a value vector characterizing the tissue;
matching the value vector to a plurality of predefined classification clusters in order to associate the tissue to be classified to an appropriate cluster, thus giving rise to a matching cluster;
transforming the value vector into a score value by use of a transformation algorithm, wherein the transformation algorithm and/or parameters thereof are selected in accordance with the matching cluster;
comparing the score value with a classification criterion corresponding to said matching cluster; and
providing a classification decision in accordance with results of said comparing.

In accordance with further aspects of the present invention, at least part of data indicative of tissue characteristics is acquired with the help of measuring the tissue characteristics by one or more sensors, as, by way of non-limiting examples, non-irradiative sensors and/or optical sensors.

In accordance with further aspects of the present invention the parameters in the classification set may be selected in accordance with one or more types of sensors used for measuring the tissue characteristics.

In accordance with further aspects of the present invention, the classification clusters are predefined in accordance with conclusive data and/or reference measurements, and may be grouped in accordance with respective tissue type and/or in accordance with respective tissue physiological features.

In accordance with further aspects of the present invention, the predefined classification clusters may be characterized by a certain sub-set of the classification parameters. Matching the value vector to a plurality of predefined classification clusters further comprises generating a value sub-vector characterized by values of parameters in said certain sub-set of the classification parameters; comparing said generated value sub-vector with said plurality of predefined clusters; and providing an automated decision with regard to a matching cluster.

Among advantages of certain embodiment of the present invention is facilitating automated real-time classification using affordable processing power.

### DESCRIPTION OF THE DRAWINGS

In order to understand the invention and to see how it may be carried out in practice, embodiments will now be described, by way of non-limiting example only, with reference to the accompanying drawings, wherein:

FIG. 1 illustrates a generalized functional block diagram of a system for tissue characterization in accordance with certain embodiments of the present invention;

FIG. 2 illustrates a generalized flow chart of a classification process in accordance with certain embodiments of the present invention;

FIGS. 3a and 3b illustrate examples of classification parameters of the acquired data characterized by a broadband resonance form;

FIG. 4 illustrates exemplary data acquired for colon tissue and clustered in accordance with certain embodiments of the present invention; and

FIG. 5 illustrates exemplary data acquired for breast tissue and clustered in accordance with certain embodiments of the present invention.

### DETAILED DESCRIPTION OF EMBODIMENTS OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, components and circuits have not been described in detail so as not to obscure the present invention. In the drawings and description, identical reference numerals indicate those components that are common to different embodiments or configurations.

Unless specifically stated otherwise, as apparent from the following discussions, it is appreciated that throughout the specification discussions utilizing terms such as “processing”, “computing”, “calculating”, “determining”, “generating”, “matching”, “transforming”, or the like, refer to the action and/or processes of a computer or computing system, or processor or similar electronic computing device, that manipulate and/or transform data represented as physical, such as electronic, quantities within the computing system's registers and/or memories into other data, similarly represented as physical quantities within the computing system's memories, registers or other such information storage, transmission or display devices.

Embodiments of the present invention may use terms such as, processor, computer, apparatus, system, sub-system, module, unit, device (in single or plural form) for performing the operations herein. This may be specially constructed for the desired purposes, or it may comprise a general purpose computer selectively activated or reconfigured by a computer program stored in the computer. Such a computer program may be stored in a computer readable storage medium, such as, but not limited to, any type of disk including optical disks, CD-ROMs, Disk-on-Key, smart cards (e.g. SIM, chip cards, etc.), magnetic-optical disks, read-only memories (ROMs), random access memories (RAMs), electrically programmable read-only memories (EPROMs), electrically erasable and programmable read only memories (EEPROMs), magnetic or optical cards, or any other type of media suitable for storing electronic instructions capable of being conveyed via a computer system bus.

The processes/devices presented herein are not inherently related to any particular electronic component or other apparatus, unless specifically stated otherwise. Various general purpose components may be used in accordance with the teachings herein, or it may prove convenient to construct a more specialized apparatus to perform the desired method. The desired structure for a variety of these systems will appear from the description below. In addition, embodiments of the present invention are not described with reference to any particular programming language. It will be appreciated that a variety of programming languages may be used to implement the teachings of the inventions as described herein.

The term “criterion” used in this patent specification should be expansively construed to include any compound criterion, including, for example, several criteria and/or their combination.

The references cited in the background are assigned to the common assignee of the present application, and teach many principles of implementation of tissue classification that are applicable to the present invention. Therefore the full contents of these publications are incorporated by reference herein, for appropriate teachings of additional or alternative details, features and/or technical background.

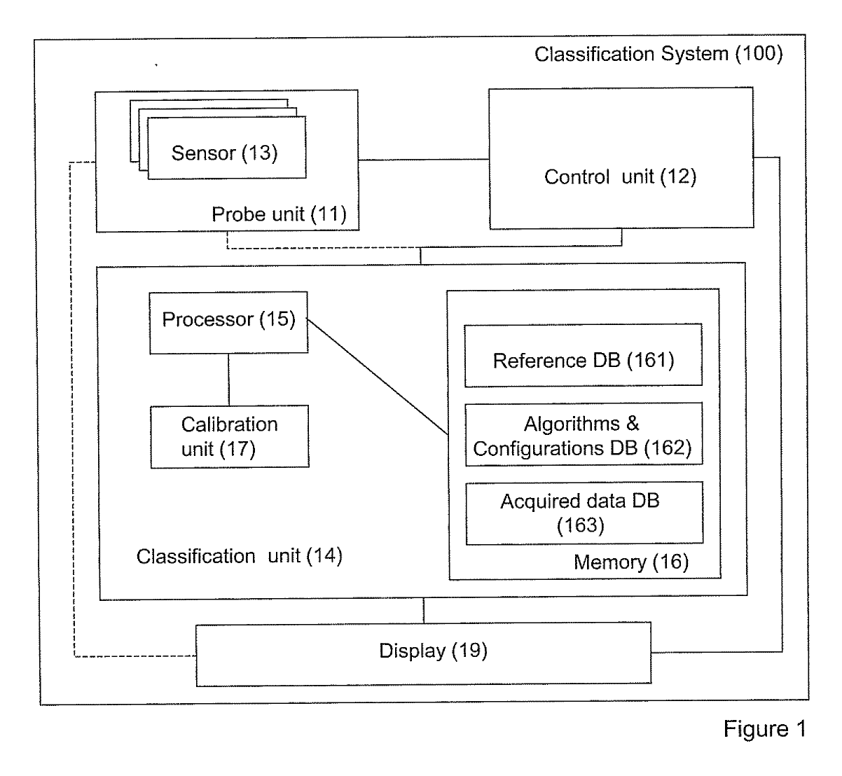

Reference is made to FIG. 1 illustrating a generalized functional block diagram of a system 100 for tissue classification in accordance with certain embodiments of the present invention. The system 100 includes a probe unit 11 operatively coupled to a control unit 12. The probe unit 11 is configured to acquire data indicative of tissue parameters, and typically includes one or more sensors 13 capable of measuring said parameters.

The probe unit and sensors thereof may be provided in different configurations, some of which are known in prior art (for example, from references cited in the background and incorporated herewith by reference). In accordance with certain embodiments of the present invention, the probe unit 11 may be configured, by way of non-limiting example, as an extracorporeal device, an intra-corporeal device, a device adapted for use on a portion of subcutaneous tissue, a device adapted for use on a portion of intra-corporeal tissue during open surgery, or otherwise. The intra-corporeal devices may be specifically configured for minimally invasive surgery and/or insertion via a trocar valve and/or for insertion via a body orifice to a body lumen (e.g. for use on a portion of an inner lumen wall for further penetrating the lumen for use on a portion of an intra-corporeal tissue outside the lumen) and/or for percutaneous insertion to a body lumen (e.g. for use on a portion of inner lumen wall for further penetrating the lumen for use on a portion of an intra-corporeal tissue outside the lumen).

According to various exemplary embodiments of the invention, the probe unit 11 is configured to obtain data indicative of one or more tissue properties as, for example, electromagnetic properties (e.g. using a non-irradiative sensor), dielectric properties, impedance, biological properties, chemical properties, optical properties (e.g. fluorescence emission and/or absorption and/or reflectance of selected wavelengths of light), MRI, energy transmission and/or reflectance (e.g. radio frequency [RF] or microwave [MW]) and temperature (e.g. via infrared thermography).

The sensors 13 may be configured, for example, as disclosed in International Published Application WO 2006/103665 entitled “Electromagnetic sensors for tissue characterization”, U.S. Pat. No. 6,813,515, entitled “Method and system for examining tissue according to the dielectric properties thereof” which are assigned to the common assignee of the present application and which is hereby incorporated by reference, or otherwise.

In some exemplary embodiments of the invention, the probe unit may include a cutting tool and/or a sampling tool.

The control unit 12 is a computer system configured to receive and transmit data and/or derivatives thereof measured by the probe unit, and to provide respective control inputs to the probe unit. The control unit typically includes inter alia a data processing utility, as well as data input and output utilities, a data storage utility, communication utility, one or more control buttons (e.g. for activating the probe unit and/or for initiating and/or measuring/classification process).

The control unit and, optionally, the probe unit are operatively coupled to a display 19 configured to display information (e.g. acquired data and/or derivatives thereof, etc.) pertinent to tissue classification, diagnosis and/or surgical planning.

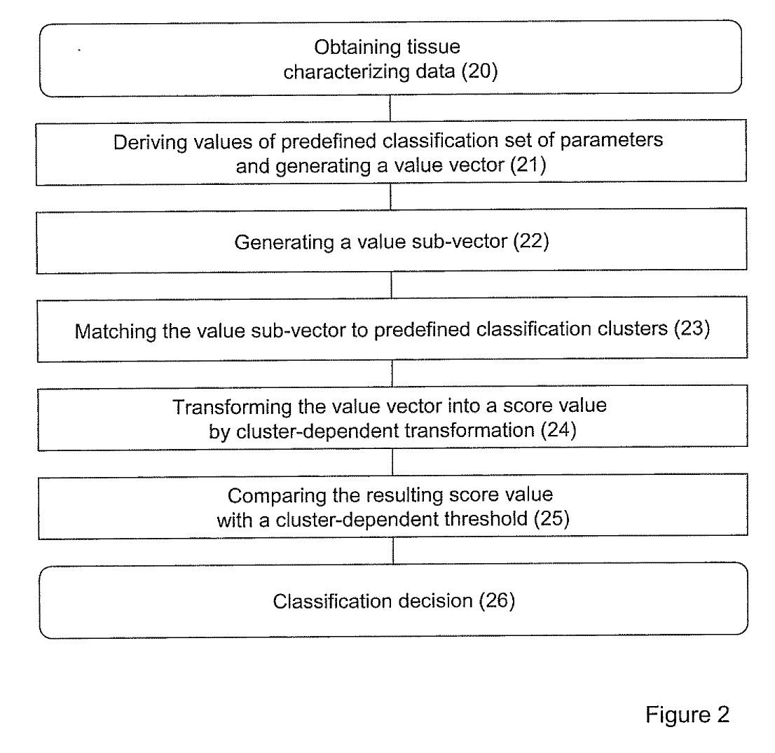

In accordance with certain embodiments of the invention, the data measured by the probe unit are classified by a classification unit 14 operationally coupled to the control unit 12, to the display 19 and to the probe unit 11 (directly or via the control unit). The classification unit is configured to receive data and/or derivatives thereof from the control unit and/or the probe unit, to analyze the received data, as will be further detailed with reference to FIGS. 2-5, and to send the results to the control unit (and/or, optionally, to the probe unit and the display). The classification unit includes a processor 15 operatively coupled to a storage memory 16 and, optionally, to a calibration unit 17. The storage memory 16 accommodates one or more data repositories, such as, for example, a database of acquired data and/or derivatives thereof 163, a reference database 161 (optional), a repository of algorithms and configurations 162 (optional), etc. The calibration unit 17 is configured to facilitate operations necessary to calibration of an measured signal.

Those versed in the art will readily appreciate that the invention is not bound by the probe unit, sensors or technique of obtaining tissue characteristics as described with reference to FIG. 1. The invention is, likewise, applicable to any probe unit configured to obtain data indicative of tissue characteristics.

Those versed in the art will also readily appreciate that the invention is not bound by the system configuration of FIG. 1; equivalent functionality may be consolidated or divided in another manner. In different embodiments of the invention the functional blocks and/or parts thereof may be implemented in dedicated physical modules and/or be distributed between different physical modules. By way of non-limiting example, the system 100 may comprise two physical modules: probe module and control module, each having its own display, while the classification functionalities may be distributed between said two physical modules. Operative connections between the physical modules and within the physical modules may be implemented directly or indirectly, including remote connection. The connection may be provided via Wire-line, Wireless, cable, Internet, Intranet or other networks and/or using any communication standard, system and/or protocol and variants or evolution thereof. The invention may also be practiced in distributed computing environments. The invention may be implemented in a stand-alone form as well as be fully or partly integrated with different devices, including 3rd party equipment.

Those skilled in the art will also readily appreciate that the data repositories may be consolidated or divided in another manner; some of these databases may be fully or partly shared with other systems, including 3rd party equipment.

Referring to FIG. 2, there is illustrated a generalized flow chart of a classification process in accordance with certain embodiments of the present invention. The data indicative of tissue parameters may be acquired (20) in different ways, some of them known in the art (e.g. measured as described in references cited in the background; as detailed in International Application IL2007/001539 filed Dec. 12, 2007 entitled “Graphical user interfaces, methods and apparatus for data presentation” and assigned to the common assignee of the present application, and which is hereby incorporated by reference and/or otherwise). The acquired data indicative of certain properties of the tissue may constitute a set of one or more single measurements at selected locations and/or may be grouped. For example, the groups may correspond to several single measurements at several locations within a certain area of the tissue and/or different measurements at the same location. In certain embodiments of the invention, a single measurement may be related to a plurality of points of the tissue measured by individual sensors belonging to a group of sensors, and provides a group of data related to the same location. In certain embodiments of the invention, the measurements (or part thereof) may be provided by a 3^{rd} party, and the respective data may be acquired from said 3^{rd} party. The acquired data may further include data indicative of tissue characteristics and obtained in a way other than measuring the tissue with a help of a sensor (e.g. demographic, clinical or other characteristics of a respective person, and the like.).

The acquired data are processed by the processor 15. This processing comprises deriving values of a first set of predefined (and/or configurable) parameters, said values being derivatives of the measured data. The first set comprises a plurality of parameters characterizing the signal. Optionally the first set of parameters (or subset thereof) may be used for signal integrity testing. The processor 15 further derives (21) values of a second set of predefined (and/or configurable) parameters. The second set of parameters is usable for tissue classification and is referred to hereinafter as a “classification set”. In certain embodiments of the invention the classification set may be the same or may constitute a sub-set of the first set and the values may be derived as a set/subset of values of the first set. In other embodiments of the invention, one or more parameters of the classification set may be other than those comprised in the first set, and their respective values may be, for example, derived from the acquired data directly (e.g. demographic, clinical or other characteristics of a respective person and the like). The values for the classification data set are derived in accordance with certain algorithms and configurations which are accommodated in the algorithms and configurations repository 162. Typically the parameters in the first set and the classification set differ for different methods of tissue characterization and/or types of sensors and/or types of organs sensed and/or clinical purposes. The derived values of the classification set are presented by a value vector characterizing the tissue to be classified. Optionally, the parameters in the classification set may have different weights and the respective values may be processed accordingly.

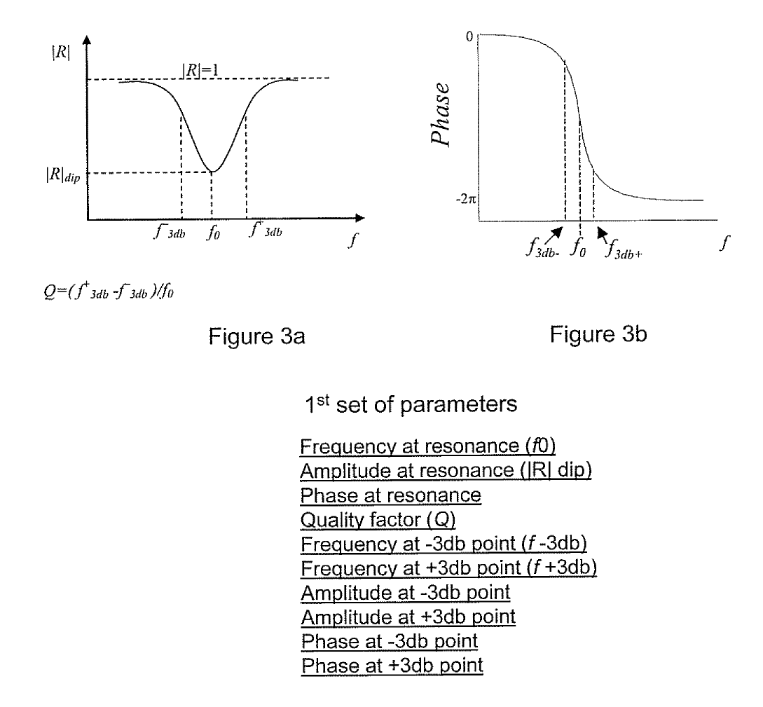

FIGS. 3a and 3b illustrate non-limiting examples of data acquired when measuring tissue by a non-irradiative electromagnetic sensor (for example such as disclosed in International Application No. WO 2006/103665). The illustrated acquired data characterize the reflection coefficient amplitude of a reflection signal (FIG. 3a) and a reflection coefficient phase of a reflection signal (FIG. 3b) depending on a frequency of the signal. Said dependency has the form of a broad-band resonator and is characterized, by way of non-limiting example, by the following parameters constituting the first set of parameters:

Frequency at resonance (fₒ)

Amplitude at resonance (|R|_{dip})

Phase at resonance

Quality factor (Q)

Frequency at −3 db point (f⁻_{3db})

Frequency at +3 db point (f⁺_{3db})

Amplitude at −3 db point

Amplitude at +3 db point

Phase at −3 db point

Phase at +3 db point

The classification sets may constitute different subsets of these parameters. When measuring the dependency between the reflection parameters and the signal frequency by non-irradiative electromagnetic sensor, the measured data for various tissue types are characterized by the broadband resonator form, while the parameters in the classification set as well as values thereof may differ, for example for different sampled organs. Additionally, as was detailed with reference to FIG. 2, one or more parameters of the classification set may be other than those comprised in the first set.

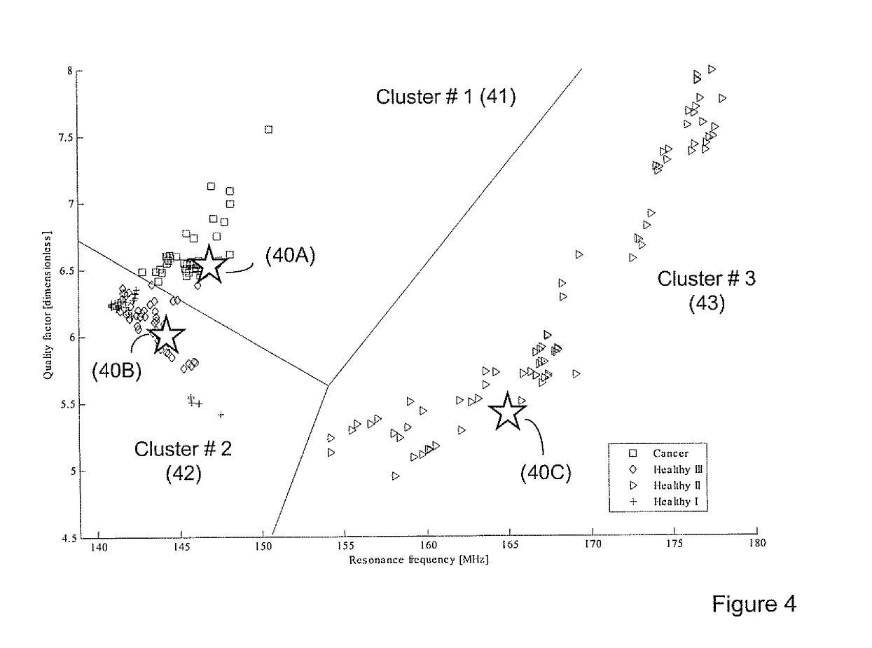

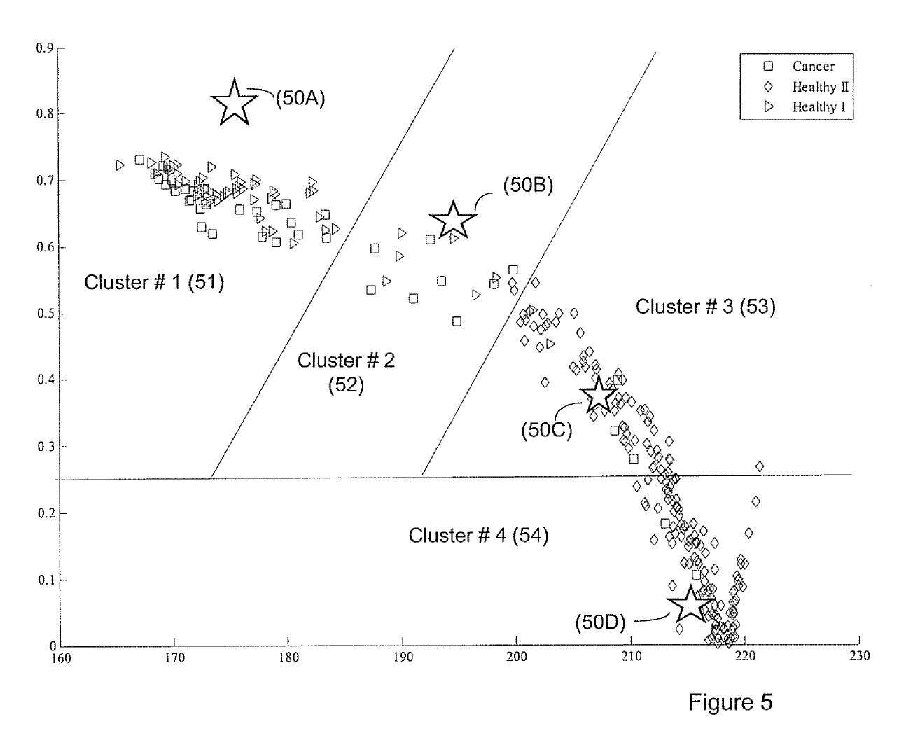

The meaningful values of parameters comprised in the classification set of parameters constitute a space of parameters. Referring back to FIG. 2, in accordance with certain embodiments of the present invention, the parameter space (or one or more sub-spaces thereof) is divided into a certain number of parameter regions configured to be mutually exclusive and collectively exhaustive. Said regions are referred to hereinafter as “classification clusters” or “clusters”. The generation of classification clusters is provided based on conclusive data (e.g., histological data from biopsy results, clinical data, and the like) and/or reference measurements. The clusters represent grouping of respective data in accordance with tissue type and/or tissue physiological features. The grouping may be provided in accordance with major tissue type/features and/or certain tissue types/features of interest and depends on the type of sensor(s) used for acquiring the data. The resulting clusters are characterized by a third set of parameters, being, typically, a sub-set of the classification set. In certain embodiments of the invention the third set may be the same as the classification set. The third set of parameters is accommodated in the algorithms and configurations repository 162. Non-limiting examples of generated clusters will be further detailed with reference to FIGS. 4-5.

The clusters' configuration is predefined before the classification process and, optionally, may be reconfigured (e.g. with the help of learning algorithms) during the process. The clusters' configuration may be accommodated in the algorithms and configurations repository 162. The processor 15 generates (22) a value sub-vector representing the values of parameters in the third set. The values may be derived as a set/sub-set of values vector or directly from the obtained data. The value sub-vector represents a relatively small set of clinically useful parameter values, and may be easily evaluated and processed. The processor compares the derived values of the third set of parameters (value sub-vector) with the predefined clusters, thus matching (23) the sub-vector to an appropriate cluster, such a cluster referred to hereinafter as a “matching cluster”. As a result, the entire respective value vector and, accordingly, the tissues characterized by said vector, are associated with the matching cluster, i.e. the cluster grouping the tissues and/or tissue features which are as similar as possible to the tissue to be classified.

The matching may be provided, for example, in accordance with minimal distance between the value sub-vector and centroids of the clusters (e.g. when using algorithms described with reference to FIGS. 4 and 5, the value sub-vector may be matched to a certain cluster based on: min{j} {sum(|aᵢpᵢ−cᵢ^{(j)}|²)}).

The processor further transforms (24) the value vector into a score value, said transformation depending on the matching cluster (e.g. by a respective cluster-dependent projection to a one-dimensional space). By way of non-limiting example, for a transformation to a MANOVA generated canonical variable, the number of parameters and/or the type of parameters and/or the weights of the parameters may be cluster dependent. By way of another non-limiting example, for nearest neighbors based transformations, the number of parameters and/or the type of parameters and/or the number of neighbors; and the metric on the distance may be cluster dependent. The transformation algorithms and/or parameters thereof are predefined (and, optionally, configurable and/or trained) for each cluster. The resulting score value is compared (25) with a respective cluster-dependent threshold value. The output of this operation serves as a basis for a classification decision (26). The thresholds may be predefined for each cluster and accommodated in the reference database 161. Alternatively, the respective thresholds may result from training a neural network based on conclusive data (e.g., histological data from biopsy results, clinical data, etc.), and associated corresponding thresholds may be accommodated in the reference database 161. Among advantages of using the score values and classification threshold is facilitating suitable classification in real-time using affordable processing power. Using a trained neural network for defining classification threshold may enhance the reliability of such a decision. Those versed in the art will readily appreciate that the invention is not bound by a one-dimensional score and threshold values, and is, likewise, applicable to other dimensions of score and/or threshold values. The number of dimensions for these values depends on the desired trade-off between exact classification and available processing power.

Optionally the classification decision may be provided in a supervised manner including comparing the value vector and/or the score value with respective values associated with corresponding conclusive data accommodated in the reference database 161. The supervision operation may take the form of searching for a look-up table, defining a nearest neighbor, training a neural network, etc.

Referring to FIGS. 4 and 5, there are illustrated non-limiting examples of generated classification clusters. In certain embodiments of the invention, the number of clusters and their boundaries correspond to distinct tissue types within certain organs and physiological characteristics thereof. The number of clusters and their boundaries may also correlate with specific features of certain tissue within the organ, which are not necessarily of a major tissue type (for example, in the prostate, benign prostatic hyperplasia tissue is composed of stroma and hyperplastic nodules, etc.). Each cluster is characterized by a centroid corresponding to a respective tissue type (marked as 40A-40C and 50A-50D respectively), and/or tissue physiological feature, within the measured organ.

In certain embodiments of the invention the clusters may be defined, for example, by use of a K-means algorithm. Parameters in which there is a correlation between the parameter values and major tissue type/features and/or certain tissue types/features of interest are selected. The selected parameters are first normalized, wherein normalization of each parameter is performed by first subtracting the average value of the parameter, and further dividing by the standard deviation of the parameter. Next, the normalized values pi are weighted with weights aᵢ. Using parameters pᵢ from a set of reference measurements, a K-means algorithm is used to find the centroids c^{(j)} relative to pᵢaⱼ. The parameters pi and weights aᵢ are adjusted until all boundaries of the clusters are as required by physiological data indications of a specific type of breast tissue, or tissue features. The operation may be provided based on conclusive data (e.g., histological data from biopsy results, clinical data, and the like), and reference measurements.

In certain embodiments of the invention the clusters may be defined, for example, by use of the parameter values of sensor readings from homogenous major tissue types and/or features. The homogenous tissues/features samples may be determined by histo-pathological analysis or otherwise. The measuring results are parameterized to a set of classification parameters. A MANOVA analysis is performed, with measurements corresponding to each tissue type assigned to a separate group. The result of the MANOVA operation generates principle/canonical variables, which are linear combinations of the classification parameters. Each group center is characterized by a specific value of the canonical variable or variables. These values of the canonical variable(s) define centroids, using the classification parameters, for each of the homogenous tissue groups selected. These centroids are used as the centroids for clustering.

FIG. 4 illustrates, by way of non-limiting example, clusters generated for colon tissue in accordance with certain embodiments of the present invention. The acquired data are presented in a phase sub-space plane and illustrate dependency between the Quality factor (Q) and the Frequency at resonance (f₀). The dependency illustrated in FIG. 4 is related to signals of a broadband resonator form detailed with reference to FIG. 3. The phase sub-space is divided into three clusters (41)-(43). The clusters differentiate between tissue which is predominantly cancerous; tissue which is predominantly healthy II (adipose tissue); tissue which is predominantly healthy I (normal) or healthy III (fibrotic). Centroids' locations are presented by stars marked respectively as 40A-40C. The third set of parameters characterizing the clusters illustrated in FIG. 4 comprises the following parameters: Quality factor (Q) and Frequency at resonance (f₀).

FIG. 5 illustrates, by way of non-limiting example, clusters generated for breast tissue in accordance with certain embodiments of the present invention. The data were acquired by a sensor with a broad-band resonator response and are presented in “Frequency at resonance”-“Amplitude at resonance” sub-space. The sub-space is divided into 4 clusters representing the regions with dominating certain type of breast tissue and/or tissue feature. These tissue types and tissue features may include: malignant invasive, malignant in-situ, mammary, connective, fibrosis, fibrocystic, adipose, adenoma. In the illustrated example the number of clusters is less than the number of tissues of interest and corresponds to a capability of distinguishing a sensor's responses with regard to different tissues. Centroids' locations are presented by stars marked respectively as 50A-50D. The third set of parameters characterizing the clusters comprises the following parameters: Frequency at resonance (f₀), Amplitude at resonance (|R|_{dip}), Frequency at −3 db point (f⁻_{3db}), Frequency at +3 db point (f⁺_{3db}), Amplitude at −3 db point.

By way of non-limiting example (not-illustrated in FIG. 5), the clusters may be defined in a one dimensional phase space by associating signals to specific clusters based on ranges of values of f₀. The ranges of f₀ may be chosen, for example, in accordance with conclusive data accommodated in the reference database 161 and indicative of a specific type of breast tissue and/or tissue features.

Those versed in the art will readily appreciate that the invention is not bound by the type of sensor and/or characteristics of acquired data illustrated with reference to FIGS. 3-5. For example, for an optically based sensor, clustering may be provided based on different absorption, scattering and fluorescence and other characteristics obtained from different tissue types. Clusters may be defined, for example, according to relative intensities in the emission maxima, frequency of the emission maxima, ratio of emission maxima at more than one wavelength, ratio of emission maxima with more than one excitation wavelength, relative intensities of absorption, relative intensities of absorption at more than one wavelength, etc. The invention is, likewise, applicable to any acquired data indicative of tissue characteristics.

Those skilled in the art will readily appreciate that the invention in some of its aspects contemplates a computer program being readable by a computer for executing the method of the invention. The invention in some of its aspects further contemplates a machine-readable memory tangibly embodying a program of instructions executable by the machine for executing the method of the invention.

It is also to be understood that the invention is not limited in its application to the details set forth in the description contained herein or illustrated in the drawings. The invention is capable of other embodiments and of being practiced and carried out in various ways. Hence, it is to be understood that the phraseology and terminology employed herein are for the purpose of description and should not be regarded as limiting. As such, those skilled in the art will appreciate that the conception, upon which this disclosure is based, may readily be utilized as a basis for designing other structures, methods, and systems for carrying out the several purposes of the present invention.

Those skilled in the art will readily appreciate that various modifications and changes can be applied to the embodiments of the invention as hereinbefore described without departing from its scope, defined in and by the appended claims.

1. A method of automated classifying a tissue, the method comprising:
a) predefining a classification set comprising one or more classification parameters;
b) acquiring data indicative of the tissue characteristics and sufficient for deriving values of said classification parameters;
c) processing the acquired data and generating a value vector characterizing the tissue, said vector characterized by values of said classification parameters;
d) matching the value vector to a plurality of predefined classification clusters in order to associate the tissue to be classified to an appropriate cluster, thus giving rise to a matching cluster;
e) transforming the value vector into a score value by use of a transformation algorithm, wherein the transformation algorithm and/or parameters thereof are selected in accordance with the matching cluster;
f) comparing the score value with a classification criterion corresponding to said matching cluster; and
g) providing a classification decision in accordance with results of said comparing.

2. The method of claim 1 wherein at least part of data indicative of tissue characteristics is acquired with the help of measuring the tissue characteristics by one or more sensors.

3. The method of claim 2 wherein at least one of the sensors is a non-irradiative sensor.

4. The method of claim 2 wherein at least one of the sensors is an optical sensor.

5. The method of claim 2 wherein the parameters in the classification set are selected in accordance with one or more types of sensors used for measuring the tissue characteristics.

6. The method of claim 1 wherein at least part of data indicative of tissue characteristics is acquired in a way other than by measuring the tissue characteristics with the help of one or more sensors.

7. The method of claim 1 wherein the parameters in the classification set have different weights.

8. The method of claim 1 wherein at least part of data indicative of tissue characteristics is acquired by measuring an electro-magnetic signal related to the tissue.

9. The method of claim 8 wherein the measured electro-magnetic signal characterizes dependency of the reflection coefficient amplitude of a reflection signal on frequency of the signal.

10. The method of claim 8 wherein the measured electromagnetic signal characterizes dependency of a reflection coefficient phase of a reflection signal on a frequency of the signal.

11. The method of claim 1 wherein the classification clusters are predefined in accordance with conclusive data and/or reference measurements.

12. The method of claim 11 wherein the classification clusters represent conclusive data and/or reference measurements grouped in accordance with respective tissue type.

13. The method of claim 11 wherein the classification clusters represent conclusive data and/or reference measurements grouped in accordance with respective tissue physiological features.

14. The method of claim 1 wherein the predefined classification clusters are characterized by a certain sub-set of the classification parameters, and said matching the value vector to a plurality of predefined classification clusters further comprises:
a) generating a value sub-vector characterized by values of parameters in said certain sub-set of the classification parameters;
b) comparing said generated value sub-vector with said plurality of predefined clusters;
c) providing an automated decision with regard to a matching cluster.

15. The method of claim 14 wherein the predefined clusters are characterized by clusters' centroids, and the automated decision with regard to a matching cluster is provided in accordance with minimal distance between the value sub-vector and the respective centroid.

16. The method of claim 1 wherein the transformation algorithms and/or parameters thereof are predefined for each cluster among the plurality of predefined clusters.

17. The method of claim 1 wherein the classification criterion is predefined for each cluster among the plurality of predefined clusters.

18. The method of claim 1 wherein at least one classification criterion results from training a neural network based on conclusive data and/or reference measurements.

19. A system for tissue characterization comprising at least one probe unit with one or more sensors, said probe unit operatively coupled to a classification unit configured to obtain data indicative of the tissue characteristics and measured by the probe unit with the help of said sensors, said classification unit comprising a processor operatively coupled to a data repository configured to accommodate a predefined classification set comprising one or more classification parameters; wherein the processor is configured to be capable of:
a) deriving from the obtained data values of said classification parameters, thus giving rise to a value vector characterizing the tissue;
b) matching the value vector to a plurality of predefined classification clusters in order to associate the tissue to be classified to an appropriate cluster, thus giving rise to a matching cluster;
c) transforming the value vector into a score value by use of a transformation algorithm, wherein the transformation algorithm and/or parameters thereof are selected in accordance with the matching cluster;
d) comparing the score value with a classification criterion corresponding to said matching cluster; and
e) providing a classification decision in accordance with results of said comparing.

20. The system of claim 19 wherein the probe unit comprises at least one non-irradiative sensor.

21. The system of claim 19 wherein the probe unit comprises at least one optical sensor.

22. The system of claim 19 wherein the parameters in the classification set are selected in accordance with one or more types of sensors used for measuring the tissue characteristics.

23. The system of claim 19 wherein the classification clusters are predefined in accordance with conclusive data and/or reference measurements.

24. The system of claim 23 wherein the classification clusters represent conclusive data and/or reference measurements grouped in accordance with respective tissue type.

25. The system of claim 23 wherein the classification clusters represent conclusive data and/or reference measurements grouped in accordance with respective tissue physiological features.

26. The system of claim 19 wherein the predefined classification clusters are characterized by a certain sub-set of the classification parameters, and for matching the value vector to a plurality of predefined classification clusters the processor is further configured to be capable of:
a) generating a value sub-vector characterized by values of parameters in said certain sub-set of the classification parameters;
b) comparing said generated value sub-vector with said plurality of predefined clusters;
c) providing an automated decision with regard to a matching cluster.

27. The system of claim 26 wherein the predefined clusters are characterized by clusters' centroids, and the automated decision with regard to a matching cluster is provided in accordance with minimal distance between the value sub-vector and the respective centroid.

28. The system of claim 19 wherein the transformation algorithms and/or parameters thereof are predefined for each cluster among the plurality of predefined clusters.

29. The system of claim 19 wherein the classification criterion is predefined for each cluster among the plurality of predefined clusters.

30. The system of claim 19 wherein at least one classification criterion results from training a neural network based on conclusive data and/or reference measurements.

31. A program storage device readable by machine, tangibly embodying a program of instructions executable by the machine to perform a method of automated classifying a tissue, the method comprising:
a) predefining a classification set comprising one or more classification parameters;
b) acquiring data indicative of the tissue characteristics and sufficient for deriving values of said classification parameters;
c) processing the acquired data and generating a value vector characterizing the tissue, said vector characterized by values of said classification parameters;
d) matching the value vector to a plurality of predefined classification clusters in order to associate the tissue to be classified to an appropriate cluster, thus giving rise to a matching cluster;
e) transforming the value vector into a score value by use of a transformation algorithm, wherein the transformation algorithm and/or parameters thereof are selected in accordance with the matching cluster;
f) comparing the score value with a classification criterion corresponding to said matching cluster; and
g) providing a classification decision in accordance with results of said comparing.

32. A computer program product comprising a computer usable medium having computer readable program code embodied therein of automated classifying a tissue, the computer program product comprising:
a) computer readable program code for causing the computer to predefine a classification set comprising one or more classification parameters;
b) computer readable program code for causing the computer to acquire data indicative of the tissue characteristics and sufficient for deriving values of said classification parameters;
c) computer readable program code for causing the computer to process the acquired data and generating a value vector characterizing the tissue, said vector characterized by values of said classification parameters;
d) computer readable program code for causing the computer to match the value vector to a plurality of predefined classification clusters in order to associate the tissue to be classified to an appropriate cluster, thus giving rise to a matching cluster;
e) computer readable program code for causing the computer to transform the value vector into a score value by use of a transformation algorithm, wherein the transformation algorithm and/or parameters thereof are selected in accordance with the matching cluster;
f) computer readable program code for causing the computer to compare the score value with a classification criterion corresponding to said matching cluster; and
g) computer readable program code for causing the computer to provide a classification decision in accordance with results of said comparing.
